(19) United States
(12) Patent Application Publication
(10) Pub. No.: US 2019/0382450 A1
(43) Pub. Date: Dec. 19, 2019

(54) NEWCASTLE DISEASE VIRUS-BASED VECTORED VACCINE

(71) Applicant: UNIVERSITY OF MARYLAND, COLLEGE PARK, College Park, MD (US)

(72) Inventors: George Belov, Rockville, MD (US); Siba K. Samal, Hyattsville, MD (US); Ekaterina Viktorova, Takoma Park, MD (US)

(21) Appl. No.: 16/443,587

(22) Filed: Jun. 17, 2019

Related U.S. Application Data
(60) Provisional application No. 62/686,480, filed on Jun. 18, 2018.

(51) Int. Cl. C07K 14/005; C12N 7/00; A61K 39/13

(52) U.S. Cl. CPC C07K 14/005; A61K 2039/575; A61K 39/13; C12N 7/00

(57) ABSTRACT

Provided are compositions and methods for vaccinating against picornaviruses. The compositions include modified Newcastle Disease viruses (NDVs) that are sufficient to produce virus-like particles (VLPs) in a host recipient. The modified NDVs contain a single stranded negative sense RNA polynucleotide having nucleotide sequences configured in a 3′-5′ direction encoding sequentially NDV nucleocapsid protein (NP), phosphoprotein (P), matrix protein (M), fusion protein (F), hemagglutinin-neuraminidase (HN) and RNA-dependent RNA polymerase (L) protein. A first nucleotide sequence encoding a picornavirus capsid polyprotein precursor is positioned between the between P and M nucleotide sequences. A second nucleotide sequence encoding a picornavirus protease that is capable of processing the capsid polyprotein precursor is positioned between the HN and L nucleotide sequences. Purified, infectious non-pathogenic NDV particles are included, as are methods for using such particles for vaccination against any infectious picornavirus. Kits and articles of manufacture containing and/or for making the NDV particles are also provided.

### CROSS REFERENCE TO RELATED APPLICATIONS

This application claims priority to U.S. provisional patent application No. 62/686,480, filed Jun. 18, 2018, the entire disclosure of which is incorporated herein by reference.

### STATEMENT REGARDING FEDERALLY SPONSORED RESEARCH OR DEVELOPMENT

This invention was made with government support under grant no. R21A1115383A awarded by the National Institutes of Health. The government has certain rights in the invention.

### BACKGROUND

New safe and effective vaccines against picornaviruses are urgently needed. For example, in the case of polio, a new vaccine is not only needed to complete the eradication, but also to be used in the future to prevent possible virus re-emergence in a post-polio world. The success of global polio eradication initiative which brought down the number of poliovirus-induced cases of paralysis from ^{˜}380000 in 1988 when it started, to less than 50 in 2016, is due to oral poliovirus vaccine (OPV). For almost entire campaign, the vaccine was deployed in its original formulation developed by Albert Sabin in the late 1950s. It contains a mixture of attenuated strains that ensures the development of robust immune response to all three serotypes of poliovirus. The most important advantages of this vaccine, are the ability to induce strong mucosal immune response necessary for interruption of the viral transmission, and low cost of its production and administration. Yet, the vaccine has important drawbacks, which became critical as the transmission of wild type viruses was diminishing. First, replication of vaccine strains may cause the disease in roughly one in a million of primary vaccine recipients (vaccine-associated paralytic poliomyelitis (VAPP)). Second, replicating viruses rapidly lose attenuating mutations and undergo recombination with other enteroviruses, thus regaining neuropathological phenotype indistinguishable from that of wild type (wt) polioviruses. Finally, at least several cases have been documented when recipients of Sabin vaccine became chronically infected with poliovirus without the development of the disease, and in some cases have been excreting the virus for decades.

To circumvent the negative consequences of live vaccine, while maintaining the adequate level of population protection, the road map of the eradication campaign calls for cessation of live vaccine use and global switch to inactivated poliovirus vaccine (IPV) by 2020. Yet, the inactivated vaccine has serious limitations of its own, most important of them being inadequate induction of mucosal immune response and high cost. The original IPV developed by Jonas Salk in the 1950s is based on formalin-inactivated wt viruses. Thus, production of such a vaccine requires handling of large quantities of pathogenic viruses, which significantly inflates the cost of the final product due to strict biosecurity measures, making it unaffordable for low-income nations.

Recently, poliovirus virus-like particles (VLPs) have been proposed as an alternative to vaccines based on inactivation of infectious virions. While VLP-based vaccines eliminate the biosafety concerns since live polioviruses are not involved at any stage of production, traditional approach based on administration of purified VLPs does not seem to be practical in case of poliovirus. Poliovirus VLPs are unstable and easily convert from protective D-antigen to inactivated C- or H-antigen conformation, which does not induce neutralizing antibodies. Even if antigenic stability of VLP can be increased by introducing mutations in the capsid protein sequence, purified VLPs are incapable of inducing mucosal immune response just like the original IPV, and scaling-up the experimental systems of VLP production and purification to the industrial level may represent a formidable challenge.

Currently, new formulations of oral vaccines as well as improvements of inactivated vaccines are being explored for use against a variety of viruses, as well as other infectious organisms, but there remains on ongoing and unmet need for an adaptable vaccine platform that can be used in a variety of settings that are not necessarily limited to controlling polio. The present disclosure is pertinent to this need.

### BRIEF DESCRIPTION OF THE FIGURES

The patent or application file contains at least one drawing executed in color. Copies of this patent or patent application publication with color drawing(s) will be provided by the Office upon request and payment of the necessary fee

FIG. 1. Recombinant NDV efficiently expresses poliovirus proteins. A. Schemes of wt NDV negative strand RNA genome and that of the recombinant virus with the poliovirus inserts. B. Western blots showing expression of NDV protein NP and poliovirus protein VP3 from the original NDV vector and NDV-polio. C. Time course of replication of the original NDV vector and NDV-polio. D. Top panel, western blots showing expression of poliovirus capsid protein VP3 in cells infected with poliovirus (lanes 1-4) and recombinant NDV-polio (lanes 5-8) at the indicated times post infection. Middle panel, the same membrane is probed with antibodies against poliovirus 3D which also recognize 3CD, and actin (loading control). Bottom panel, the same western blot as in the middle panel after longer exposure to better visualize expression of 3CD in cells infected with recombinant NDV-polio virus.

FIG. 2. Polio VLPs are efficiently produced in cells infected with recombinant NDV-polio virus. A. Immunofluorescent staining using monoclonal A12 antibodies recognizing conformational epitope in fully formed poliovirus capsid of cells infected with recombinant NDV-polio virus (left panel), poliovirus (middle panel) and mock-infected cells (right panel). Nuclei are visualized with a DNA stain Hoechst 33342. B. Transmission EM image of a HeLa cell infected with NDV-polio. M—mitochondrion, ER—endoplasmic reticulum, VLP—polio virus-like particles. C. Scheme of the polio replicon transecapsidation experiment using capsid proteins expressed from recombinant NDV-polio virus. D. C. Renilla luciferase kinetics curves showing strong polio replication only in samples treated with lysates of cells infected with NDV-polio and transfected with polio replicon RNA, confirming effective transcapsidation of the replicon RNA by poliovirus capsid proteins expressed from recombinant NDV-polio virus.

FIG. 3. Recombinant NDV-polio virus is stable upon propagation in embryonated eggs. A. Double immunostaining of cells infected with recombinant NDV-polio virus with a mouse monoclonal antibodies recognizing an NDV antigen HN and a chimpanzee-human hybrid monoclonal antibody A12 recognizing a conformational epitope in fully-assembled poliovirus capsid. Nuclei are visualized with a DNA stain Hoechst 33342. B. Quantitation of double-positive cells as in A, indicative of accumulation of correctly assembled poliovirus capsids in cells infected with recombinant NDV-polio viruses propagated for 10 passages in embryonated chicken eggs. Several fields containing together at least 100 cells were quantified. C. Western blot of lysates from HeLa cells infected with an MOI of 10 PFU/cell of recombinant NDV-polio viruses propagated for 1 or 10 passages in embryonated chicken eggs. Expression of the poliovirus capsid protein VP3 and NDV protein NP is shown. Actin shown as a loading control.

FIG. 4. Intranasal immunization with recombinant NDV-polio virus induces systemic and mucosal anti-poliovirus response. A. The development of anti-NDV IgG response upon intranasal immunization of Guinea pigs with either recombinant NDV-polio virus or the NDV LaSota strain virus without polio inserts. B. The development of anti-poliovirus neutralizing antibody response, or anti-poliovirus IgM and IgA in guinea pigs immunized intranasally with recombinant NDV-polio virus. Titers are expressed as reciprocal number of the serum dilution shown on a Log₂ scale. Red dashed line on the neutralization antibody panel indicates 1:8 serum titer considered sufficient for anti-poliovirus protection. C. The development of anti-poliovirus IgG and neutralizing antibodies in vaginal washes of guinea pigs immunized with recombinant NDV-polio virus. Titers are expressed as reciprocal number of the serum dilution shown on a Log₂ scale.

### SUMMARY

The present disclosure provides a novel design of a picornavirus vaccine. Without intending to be constrained by any particular theory, it is considered that the disclosure combines the benefits of a live vaccination approach with the safety of VLPs.

In embodiments, the disclosure comprises expression vectors that are configured to express VLPs in the organisms of vaccine recipients, using, for example, a viral vector with mucosal tropism. Such system does not require purification and stabilization of liable VLPs, and they are presented to the immune system in the context of active viral replication, which serves as a natural adjuvant and promotes the development of a strong mucosal immune response. Thus, in embodiments, the disclosure provides compositions comprising infectious, non-pathogenic viral particles as described herein, wherein the composition is adjuvant free, other than the viral particles themselves having an adjuvant function by way of VLP production in the host recipient. In non-limiting examples, adjuvants that can be excluded from the present disclosure include alum, AS04, pathogen components, such as Monophosphoryl lipid A (MPL), Poly(I:C), which is a synthetic double-stranded RNA that mimics a molecular pattern associated with viral infection, CpG DNA, which are short segments of DNA that include sequence motifs, or patterns, commonly found in bacterial DNA, and emulsions, e.g., a blend of two liquids that are normally unmixable, such as water and oil, one example of which is MF59. Additional adjuvants that can be excluded from compositions of this disclosure also include, but are not limited to, virosomes and cytokines. Combinations of adjuvants can also be excluded.

As a non-limiting demonstration, we introduced sequences coding for poliovirus capsid protein precursor P1 and protease 3CD necessary for its processing into the genome of a non-pathogenic strain NDV. NDV is easily administered via nasal and oral routes and has been shown to elicit strong systemic and mucosal immune responses, including gut mucosal immunity in murine and nonhuman primate models.

It will be recognized from data presented herein that the disclosure provides a novel design of a superior anti-picornavirus vaccine, which can be adopted with minimal modifications to combat any picornavirus infections, since all picornaviruses share the same polyprotein expression strategy and require a protease for processing of the capsid protein precursor. Accordingly, in embodiments, the present disclosure provides vectored NDV-based vaccines which combine economic efficiency of a live attenuated vaccine with the safety of a VLP-based vaccine, and a novel scheme of protein co-expression for optimization of picornavirus VLP production. Taking advantage of a gradient transcription pattern of NDV genes, the presently provided vaccines express high levels of, for example, the capsid protein precursor and low level of a cytotoxic protease necessary for its processing. Since as described above NDV-based vaccines against picornaviruses can be delivered intranasally, significant reduction of cost of vaccine administration is provided, which is especially important for veterinary vaccines, and in limited resource countries. NDV-based vaccines can be propagated in embryonated chicken eggs, a highly efficient economic system, significantly outperforming traditional systems for picornavirus vaccine production, such as cell culture or recombinant protein technology. The presently provided vaccine does not require propagation of live picornaviruses at any stage, thus significantly reducing biosafety risks and associated costs.

### DETAILED DESCRIPTION

Unless defined otherwise herein, all technical and scientific terms used herein have the same meaning as commonly understood by one of ordinary skill in the art to which this disclosure pertains.

Unless specified to the contrary, it is intended that every maximum numerical limitation given throughout this description includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All nucleotide sequences described herein include the RNA and DNA equivalents of such sequences, i.e., an RNA sequence includes its cDNA. All nucleotide sequences include their complementary sequences.

All nucleotide and amino acid sequences identified by reference to a database, such as a GenBank database reference number, are incorporated herein by reference as the sequence exists on the filing date of this application or patent.

The disclosure includes all embodiments illustrated in the Figures provided with this disclosure. In certain embodiments, the disclosure is related to production of VLPs in a host. As is known in the art, VLPs are particles that include viral proteins, but do not include the viral genome.

In certain embodiments, the disclosure provides one or more modified NDVs that are produce VLPs in a host recipient. The modified NDV(s) comprises a single stranded (ss) negative sense RNA polynucleotide adapted from an NDV genome. The ssRNA comprises nucleotide sequences configured in a 3′-5′ direction which encode, sequentially, NDV nucleocapsid protein (NP), phosphoprotein (P), matrix protein (M), fusion protein (F), haemagglutinin-neuraminidase (HN) and RNA-dependent RNA polymerase (L) protein.

By stating a negative sense ssRNA polynucleotide “encodes” (and its various grammatical forms), it is meant that the ssRNA directs L protein-mediated transcription of a complementary RNA from which the described proteins are translated. The ssRNA NDV further comprises: i) a first nucleotide sequence encoding a picornavirus capsid polyprotein precursor, the first nucleotide sequence positioned between the between P and M nucleotide sequences, and ii) a second nucleotide sequence encoding a picornavirus protease that is capable of processing the capsid polyprotein precursor such that the individual, processed capsid proteins are assembled into the VLPs. The second nucleotide sequence is positioned between the HN and L nucleotide sequences.

The functional relationship between picornavirus proteases and capsid proteins, including capsid polyprotein precursor proteins, in picornavirus VLP production cycle is well known to those skilled in the art. A non-limiting demonstration of the general approach of the disclosure is provided using poliovirus type I Mahoney capsid protein precursor P1, and poliovirus protease 3CD. The same approach can be adapted using any matched picornavirus capsid precursor protein and protease pair. For example, the presently provided vaccine production scheme can be adapted for use to include, and thereby vaccinate against, capsid proteins from any picornavirus. The disclosure thus includes incorporating into the NDV-based vaccines a capsid protein and a protease that processes the capsid protein from any infectious member of the Picornaviridae family. Many examples of infectious members of this family are well known in the art. In embodiments, the disclosure includes any capsid polyprotein precursor/protease pair that is known in the art as of the effective filing date of this application or patent. In embodiments, the capsid polyprotein precursor/protease pair is from any picornavirus that causes poliomyelitis, encephalitis, meningitis, hepatitis, non-polio flaccid paralysis, Foot and mouth disease, or the common cold. In embodiments, the disclosure includes use of any capsid polyprotein precursor/protease pair in the modified NDVs of this disclosure that are from any infectious member of the following genera:

Ampivirus, Aphthovirus, Aquamavirus, Avihepatovirus, Avisivirus, Cardiovirus, Cosavirus, Dicipivirus, Enterovirus, Erbovirus, Gallivirus, Harkavirus, Hepatovirus, Hunnivirus, Kobuvirus, Kunsagivirus, Limnipivirus, Megrivirus, Mischivirus, Mosavirus, Oscivirus, Parechovirus, Pasivirus, Passerivirus, Potamipivirus, Rabovirus, Rosavirus, Sakobuvirus, Salivirus, Sapelovirus, Senecavirus, Sicinivirus, Teschovirus, Torchivirus, and Tremovirus.

In certain embodiments, the disclosure relates to a capsid polyprotein precursor/protease pair that is from any infectious member of Enterovirus, non-limiting examples of which include the aforementioned poliovirus, as well as rhinovirus, coxsackievirus, and echovirus. In specific and non-limiting examples, the capsid/protease pair is from Enterovirus 71, a significant current public health threat in Asia-Pacific region associated with multiple infant deaths, or from Enterovirus D68, an emerging pathogen associated with non-polio flaccid paralysis cases in the USA, and Foot and mouth disease virus, a major threat to animal husbandry worldwide.

The capsid polyprotein precursor and protease coding sequences can be flanked by any suitable regulatory sequences, such as NDV transcription start and stop signals. In embodiments, the NDVs have mucosal tropism. In an embodiments, a non-pathogenic NDV that is used to create the vaccines described herein comprises a LaSota strain of NDV.

In certain aspects the disclosure includes a pharmaceutical formulation comprising modified NDV infectious, non-pathogenic particles (mNDV-NPPs). The form of pharmaceutical preparation is not particularly limited, but generally may comprises mNDV-NPPs and at least one inactive ingredient. In certain embodiments suitable pharmaceutical compositions can be prepared by mixing any type of mNDV-NPP, or combination of distinct mNDV-NPPs, with a pharmaceutically-acceptable additive, such as a stabilizer, carrier, diluent or excipient, or immune response regulator, and suitable such components are well known in the art. Some examples of such carriers, diluents and excipients can be found in: Remington: The Science and Practice of Pharmacy (2005) 21st Edition, Philadelphia, Pa. Lippincott Williams & Wilkins, the disclosure of which is incorporated herein by reference. Non-limiting examples of suitable stabilizers include gelatin, sucrose, sodium glutamate, calcium alginate beads, Vitamin E TPGS, Carbopol®, poly(2-ethyl-2-oxazoline), chelating agents such as ethylenediaminetetraacetic acid (EDTA), any of a variety of surfactants, etc.

mNDV-NPPs can be made using standard techniques, when given the benefit of the present disclosure. Representative and non-limiting examples of mNDV-NPPs production is described further below in the Examples. The mNDV-NPPs can also be purified using standard approaches, and may be purified to any desired degree of purity. In embodiments, the mNDV-NPPs may be provided, for example, in a lyophilized or freeze dried form for reconstitution before administration.

Compositions of this disclosure comprising mNDV-NPPs can be administered using standard techniques. In embodiments, the mNDV-NPPs are administered to a mammal or an avian animal using any suitable route. In embodiments, the mNDV-NPPs are provided as vaccines and can be administered orally, or by intramuscular or subcutaneous injection. In embodiments, the vaccine formulations are administered via an oculanasal route, and thus both conjunctival and intranasal routes are included. In embodiments, the vaccine is administered as an aerosol or a spray. In embodiments, any mucosal administration is used.

Dosing of the compositions of this invention can be determined based on known parameters, such as the age, size/weight, and gender of the individual, and the type of picornavirus infection the individual has or is at risk for developing. In embodiments, an immunologically effective amount of mNDV-NPPs is administered. “Immunologically effective” as used herein means an amount that results in production of neutralizing antibodies against the particular picornavirus from which the capsid and protease are used, and/or results in reduced shedding of the challenge virus, or a reduction in clinical signs of infection, or protection against mortality caused by the infection of the particular picornavirus. In embodiments, neutralizing antibodies are produced. The term “neutralizing antibody” and its various grammatical forms refers to antibodies that inhibit, reduce or completely prevent viral infection. Whether neutralizing antibodies are produced can be determined by in vitro assays that are known in the art. In embodiments, viral load in the vaccinated animals is reduced. Viral load can also be determined according to methods known to those skilled in the art. In embodiments, an embryo infective dose (EID) is used.

Immunological protection elicited by the presently provided vaccines can be durable, and last for days, weeks or months, or longer, after vaccination, and such vaccinations can be effective to elicit such protection after a single dose, or multiple doses. Booster vaccinations can be used, if desired.

In embodiments, a composition of the disclosure is administered to a mammal, such as a human, or a non-human mammal. In embodiments, the individual has contracted, or is at risk of contracting, a picornavirus infection.

Non-limiting examples of non-human mammals which can benefit from the provided vaccines include ruminants, including but not necessarily limited to bovines, sheep, antelopes, deer, giraffes, and their relatives, and further can include pseudoruminants, such as the camelids. In embodiments, the ruminant is bovine mammal that is a member of the genus Bos, such as oxen, cows, and buffalo. In one embodiment the ruminant is a dairy cow.

In an embodiment the disclosure includes administering a composition of this disclosure to a member of the genus Sus, and therefore encompasses practicing the invention with any swine, examples of which are not limited to the domestic pig (i.e., Sus domesticus), also commonly referred to as a swine or a hog.

The disclosure also includes administering the compositions to non-bovine and non-ruminant mammals, including but not necessarily limited to equines, canines, and felines. Thus, the disclosure in certain aspects pertains to vaccination of companion animals, as well as animals kept in conservation settings, for example in zoos.

In embodiments, avian animals to which compositions of this disclosure are administered are any type of poultry. In embodiments, the avian animals are Galliformes and thus include any members of the order of heavy-bodied ground-feeding birds that includes turkey, grouse, chicken, New World quail and Old World quail, ptarmigan, partridge, pheasant, junglefowl and the Cracidae. In embodiments, the avian animals are domesticated fowl, including but not limited to domesticated chickens and turkeys. In embodiments, the chickens are Gallus gallus, such as Gallus gallus domesticus. In embodiments, the chickens are roosters or hens. In embodiments, the avian animals are adults, juveniles, or embryos. In an embodiment, a composition of this disclosure is applied to eggs. In embodiments, vaccines of this disclosure administered to a population of avian animals, i.e., a flock. In embodiments, from 50-85% or more members of the flock are vaccinated to achieve, for example, herd or flock immunity; this approach applies to mammals as well.

In embodiments, the disclosure includes an article of manufacture, such as a kit, the kit comprising mNDV-NPPs as described herein in any suitable form, wherein the mNDV-NPPs are comprised within one or more containers, and wherein the article or kit comprises printed material, such as a label or insert that includes instructions for using the mNDV-NPPs for vaccination of mammals or avian animals. In embodiments, a kit comprises a modified NDV genome cDNA equivalent, wherein the cDNA comprises a set of two distinct restriction cloning sites for inserting a picornovirus capsid and protease coding sequence.

The following Examples are intended to illustrate, but not limit the disclosure.

### Example 1

Construction of an NDV Vector Expressing Poliovirus Proteins.

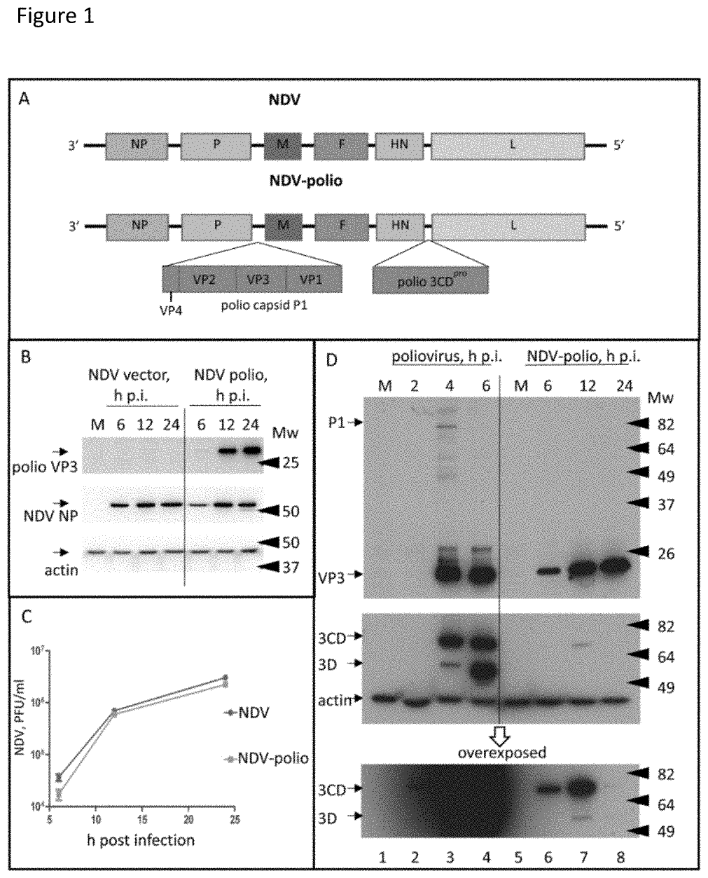

NDV genome contains six genes in the order 3′-NP-P-M-F-HN-L-5′ (FIG. 1A). Transcription of each gene is controlled by start and stop signals so that the viral RNA-dependent RNA polymerase performs consecutive transcription initiation-termination cycles moving from the 3′ end of the genome. Since re-initiation efficiency is not absolute, this strategy results in transcription gradient where the genes located closer to the 3′ end of the genome are expressed at a higher level than those closer to the 5′ end. We introduced the sequence coding for poliovirus type I Mahoney capsid protein precursor P1 between P and M genes, and that coding for poliovirus protease 3CD between HN and L genes of a non-pathogenic LaSota strain of NDV (FIG. 1A). Both poliovirus inserts were engineered to be flanked by NDV transcription start and stop signals. This design was chosen to provide a high level of expression of P1 structural protein precursor and a relatively low level of expression of 3CD protease, minimizing its possible toxic effect on cellular metabolism. Recombinant NDV was successfully rescued, and demonstrated growth properties similar to those of the original virus without polio inserts (FIGS. 1B and C). To characterize the level of poliovirus protein expression, HeLa cells were infected with 10 PFU/cell of NDV-polio and the lysates were collected at 6, 12 and 24 h post infection (p.i.). By 24 h p.i. NDV-induced CPE becomes prominent and the cells begin to die extensively. For comparison we used lysates from HeLa cells infected with 10 PFU/cell of poliovirus type I Mahoney, collected at 2, 4 and 6 h p.i.; by 6 h replication of poliovirus in HeLa cells is complete. The amount of VP3 and 3CD produced by recombinant NDV reached its maximum level by 12 h p.i. Interestingly, the level of the capsid protein remained the same in the sample collected at 24 h p.i., while that of 3CD decreased significantly, so that only traces of the protein could be detected at 24 h p.i. (FIG. 1D, NDV-polio samples). This peculiar pattern may reflect the position of 3CD close to the 5′ end of the genome, so that 3CD expression regulation may be similar to its neighboring L gene, a viral RNA-dependent RNA polymerase. It is likely that the polymerase expression is inhibited towards the end of infection. It may also be due to a different stability of structural polio proteins assembled into stable VLPs and a 3CD protein. The level of 3CD expression from recombinant NDV was much lower than that in poliovirus-infected cells, as was predicted from the design of the recombinant construct (FIG. 1D, 3CD panels). Interestingly, this lower level of 3CD was sufficient for complete processing of P1 in NDV-polio infected cells even at the earliest time point sampled, while in poliovirus-infected samples the unprocessed P1 precursor and numerous cleavage products could be detected in spite of much higher level of 3CD expression (FIG. 1B). Thus, poliovirus capsid precursor P1 and protease 3CD can be efficiently co-expressed as separate genes from a recombinant NDV vector, resulting in a robust processing of P1 into individual capsid proteins.

### Example 2

Poliovirus Capsid Proteins Expressed by Recombinant NDV are Fully Functional and Assemble into VLPs.

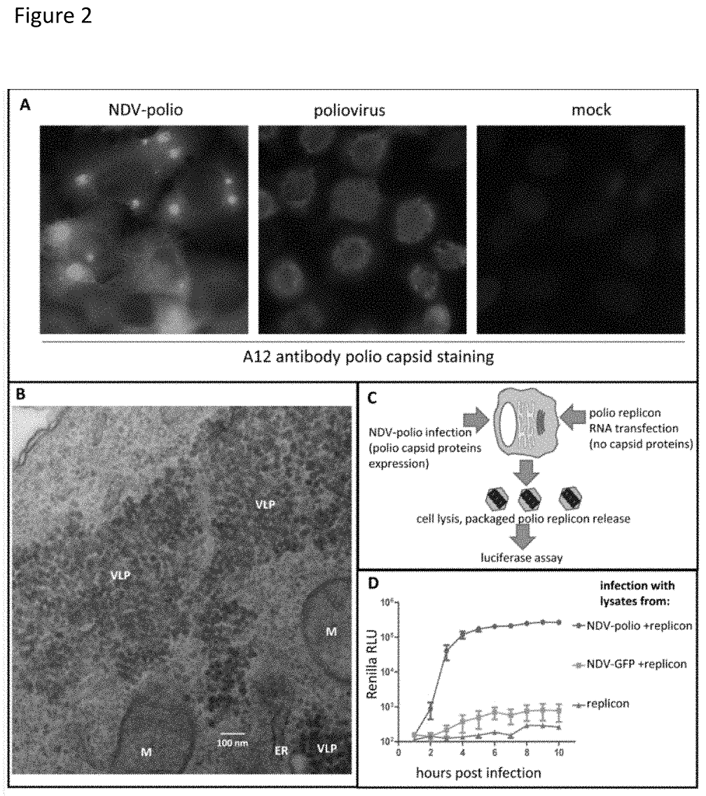

To see if poliovirus capsid proteins assemble into VLPs we performed immunofluorescent assay of cells infected with NDV-polio with a monoclonal antibody A12, which recognizes a conformational epitope present only in fully assembled poliovirus capsids. HeLa cells infected with NDV-polio were fixed at 12 h p.i., for control we used poliovirus-infected HeLa cells fixed at 6 h p.i. Cells infected with recombinant NDV-polio demonstrated massive A12-specific signal, indicating strong accumulation of the relevant poliovirus antigen (FIG. 2A). While in control poliovirus-infected cells the capsid signal was localized in perinuclear rings, typical for localization of the replication complexes, in recombinant NDV-infected cells the staining was more diffuse, it occupied the whole cell volume and was often highly concentrated in several spots per cell (FIG. 2A). An electron microscopy examination of the cells infected with NDV-polio revealed large clusters of polio VLPs in the cytoplasm, which likely correspond to the bright spots detected in the immunofluorescence assay (FIG. 2B).

To observe if poliovirus capsid proteins expressed from recombinant NDV could form functional poliovirus capsids we performed a transencapsidation assay of polio replicon RNA. Poliovirus capsid proteins encoded in the 5′ end of the genome RNA are dispensable for RNA replication. Capsid protein coding sequence can be substituted with a reporter protein gene so that such an RNA would be fully functional in replication but will be incapable to form infectious virions, unless functional capsid proteins are provided in trans. We infected HeLa cells with 10 PFU/cell of NDV-polio and transfected them with a poliovirus replicon RNA with the Renilla luciferase gene in place of capsid coding sequence. In control samples the cells were infected with NDV-GFP, a recombinant NDV expressing fluorescent proteins instead of polio inserts, or transfected with the polio replicon RNA alone. About 20 h post transfection with polio replicon RNA the cells were freeze-thawed three times to release any packaged replicon (FIG. 2B). To assess the presence of a packaged replicon, these lysates were used to infect HeLa cell monolayer and the Renilla luciferase signal was monitored over 12 h. Strong replication signal confirmed that infectious virions containing polio replicon RNA were efficiently formed in cells infected with NDV-polio and transfected with replicon RNA (FIG. 2C). Collectively, these data demonstrate that co-expression of poliovirus capsid protein precursor P1 and protease 3CD from a recombinant NDV vector produces a high level of poliovirus VLPs with antigenic properties of fully functional virions.

### Example 3

Propagation and Stability of Recombinant NDV in Embryonated Chicken Eggs.

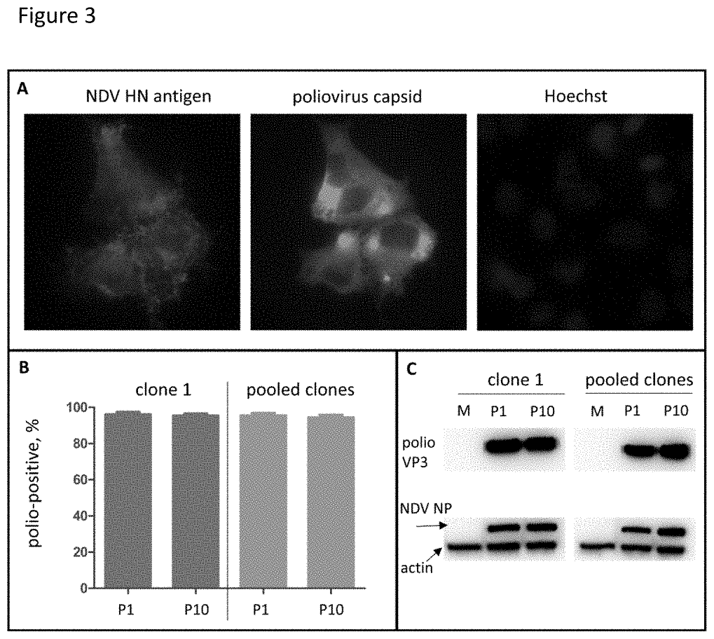

An important characteristic of a recombinant vaccine vector is the ability to retain the foreign insert(s) over multiple replication cycles. To determine the stability profile of the NDV-polio construct we first plaque-purified 10 individual clones of NDV-polio, and individually passaged clone 1 and a mixture of all 10 individual clones for 10 times in embryonated eggs. To assess the percentage of viruses expressing poliovirus VLPs HeLa cells were infected with a low MOI of ^{˜}0.5 PFU/cell of the viruses from passages 1 and 10, so that the individually infected cells would be clearly separated from each other. The non-pathogenic LaSota strain of NDV used as a backbone for the recombinant construct cannot form infectious virions in the absence of exogenously added proteases, and can spread only to cells in direct contact with the originally infected one, producing foci of a few adjacent infected cells. The next day the cells were fixed and double stained with antibodies specific for an NDV antigen, and antibody A12 recognizing a conformational epitope in poliovirus capsid (FIG. 3A). In the cells infected with either individual clone 1 or pooled clones the expression of polio capsid proteins was detected in ^{˜}95% of NDV-positive cells and this percentage remained essentially the same after 10 passages (FIG. 3B). We also compared the expression of poliovirus capsid protein VP3 in a western blot analysis of HeLa cells infected with individual clone 1 and pooled clones passaged for one and 10 times in embryonated eggs. The level of VP3 expression did not change significantly, neither we observed any other VP3-positive bands that may indicate possible rearrangements/deletions within the poliovirus inserts (FIG. 3C). Finally, to characterize possible mutations upon serial passages, the viral RNA isolated from the pooled material at passages 2 and 11 (we performed additional passages for propagation of sufficient amount of material for RNA isolation) was subject to next generation sequencing (NGS). The analysis of NGS data with the HIVE software package did not reveal any significant accumulation of point mutations or the viral genomes with deletions/rearrangements of the poliovirus inserts (the data are deposited at hive.biochemistry.gwu.edu/review/NDV_LaSota_strain_PV), in agreement with the biochemical and cytological assays.

These data show that recombinant NDV-polio construct is highly stable upon propagation in embryonated chicken eggs, an established and economically attractive system of vaccine production.

### Example 4

Intranasal Administration of NDV-Polio Induces Neutralizing and Mucosal Anti-Poliovirus Antibodies in Guinea Pigs.

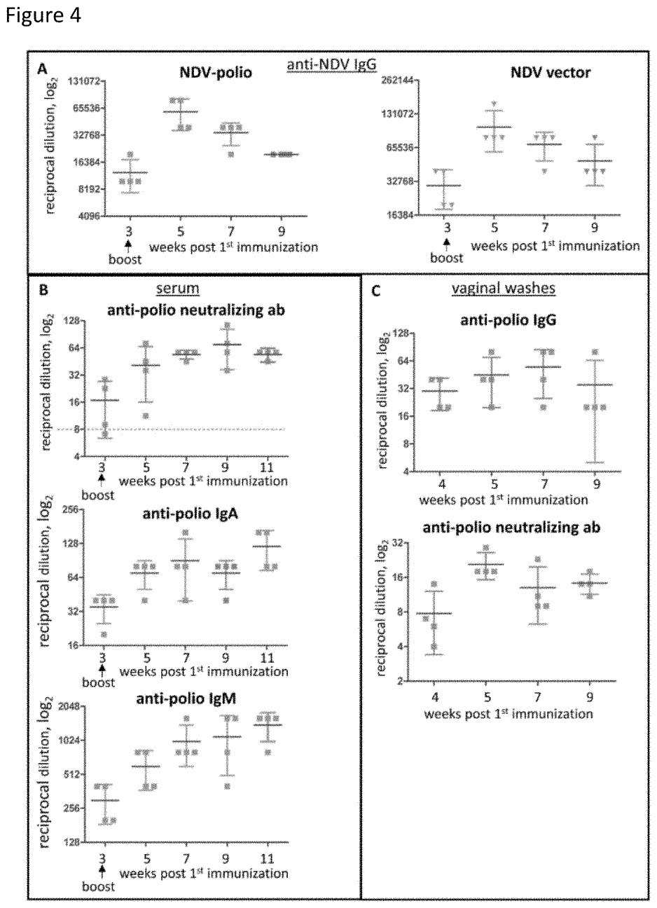

To evaluate the efficacy of anti-poliovirus vaccination with NDV-polio construct we performed intranasal immunization of guinea pigs. Previous research indicates that immune response of guinea pigs to NDV vector-based vaccines resembles that observed in primate models. Four animals in each group were immunized with either NDV-polio or NDV vector without poliovirus inserts. Booster immunization was performed three weeks after the first immunization, and blood serum as well as vaginal washes were collected before immunization and on weeks 3, 4, 5, 7, 9 and 11 post immunization. The animals did not show any adverse effects associated with immunization, as was observed previously even for multiple cycles of administration of NDV-based vaccines. Replication of NDV was confirmed in all animals as evidenced by the development of serum anti-NDV IgG response. The level and the dynamics of accumulation of anti-NDV antibodies was similar in all animals with a clear boost after the second immunization, indicating that NDV with polio inserts is not compromised compared to the parental virus in vivo. (FIG. 4A). To assess the level of neutralizing anti-poliovirus antibodies we performed a microneutralization assay, which is a standard tool for evaluation of the efficacy of anti-poliovirus vaccination. All the animals immunized with NDV-polio showed the development of neutralizing antibody response. One serum sample collected at week 3, before the booster immunization, showed the level of neutralizing antibodies slightly below the 1:8 threshold, generally accepted as sufficient for protection against poliomyelitis. Nevertheless, the level of neutralizing antibodies in samples collected at week 7 and after was well above the protective threshold in all the animals (FIG. 4B, neutralizing antibodies panel). The highest serum dilutions exhibiting neutralizing activities ranged from ^{˜}1:35 to ^{˜}1:100 in different animals, and remained relatively stable at least until week 11 post immunization (FIG. 4B, neutralizing antibodies panel). To evaluate the development of mucosal immune response we first measured the level of IgM and IgA antibodies in the serum of immunized animals. IgM is the first antibody class to appear during the development of immune response and they also contribute to the mucosal immunity, and IgA is the main antibody class expressed on mucosal surfaces. Presence of IgM and IgA in serum was shown to correlate with mucosal immune response including that in the gut in many systems. The serum was assessed for the presence of antibodies capable of binding poliovirus type I virions from a standard IPV preparation bound to a nitrocellulose membrane. The samples from animals immunized with NDV-polio showed a steady accumulation of anti-poliovirus IgM and IgA which reached its peak at 7 weeks post immunization and stayed relatively stable after that (FIG. 4B, IgM and IgA panels). As expected, no neutralizing or mucosal anti-poliovirus antibody response was detected in control group immunized with NDV without poliovirus inserts. To assess the development of bona fide mucosal anti-poliovirus response, we evaluated the level of poliovirus-specific antibodies in vaginal washes. For correct comparison, the protein concentration in vaginal wash samples collected at different time points and from different animals was adjusted to the same level with PBS before titration. Some vaginal washes contained very little protein, likely because of an estrus cycle stage, and were excluded from the analysis. Since anti-guinea pig IgA secondary antibody conjugate gave a very high background with vaginal wash samples, we assessed the level of secreted anti-poliovirus IgGs, these antibodies are among the most abundant in urogenital tract secretions. They peaked at 4 and 5 week post-immunization samples, and decreased significantly in all but one animal by week 9 (FIG. 4 C). In a microneutralization assay anti-polio neutralizing activity was detected in vaginal washes from all animals immunized with NDV-polio, but not from the control group immunized with NDV without polio insert. It peaked at week 5 and remained relatively stable up to week 9 (FIG. 4C). Thus, intranasal immunization with recombinant NDV-polio virus induces robust protective anti-poliovirus systemic and mucosal response.

### Example 5

The following Materials and Methods were used to provide the above-described results.

Cells and Viruses.

Human cervical carcinoma Hela cell line was received a third party; a HeLa-derivative HEp-2 cell line and chicken embryo fibroblast DF-1 cells were from ATCC. HeLa cells were maintained in DMEM high glucose modification medium supplemented with pyruvate and 10% FBS. HEp-2 cells were grown in MEM supplemented with 10% FBS. (DF1) were maintained in DMEM supplemented with 10% FBS. Poliovirus type I Mahoney was propagated in HeLa cells and the viral titer was determined either by plaque assay and expressed by PFU/ml (for experiments where expression of poliovirus proteins was assessed), or by terminal dilution method and expressed as TCID₅₀/ml calculated by Karber's formula for assessment of neutralizing antibodies. Recombinant NDV was rescued using reverse genetic system according to known approaches with minor modifications. Briefly, HEp-2 cells were infected with vaccinia virus expressing T7 RNA polymerase and transfected using Mirus 2020 transfection reagent with three plasmids coding for NDV NP, P and L genes (polymerase complex), as well as the plasmid coding for the full length NDV genome with the poliovirus inserts, under control of T7 promotor. The cells were incubated in MEM medium supplemented with 10% of fresh allantoic fluid for two days post transfection. After that, the cells were subjected to three cycles of freeze-thawing, the medium was collected, clarified from the cellular debris by low speed centrifugation and used to inoculate allantoic cavity of 9 days old chicken embryos. After 48 hours allantoic fluid was collected and tested for the presence of haemagglutinating (HA) activity, indicative of NDV propagation. Virus titer was determined by plaque assay on DF1 cells overlaid with methylcellulose-solidified growth medium supplemented with 10% of fresh allantoic fluid for 3 days.

Plasmids.

pLaSota plasmid coding for the full length cDNA of LaSota strain of NDV as well as the plasmids coding for the LaSota polymerase complex genes N, P and L under control of T7 promotor have been described. Plasmid pXpA coding for the full length cDNA of poliovirus type I Mahoney has been described. Plasmid pNDV-polio was constructed by inserting PCR-amplified fragments coding for poliovirus capsid protein precursor P1 and protease 3CD in PmeI site located between the P and M genes, and SnaBI site located between the HN and L genes of LaSota sequence, respectively. Poliovirus P1 and 3CD inserts were designed to be flanked with transcription initiation and termination signals for LaSota polymerase. Details of primer design and other cloning considerations are available upon request.

Antibodies.

Anti-poliovirus VP3 mouse monoclonal antibody were obtained separately, anti-poliovirus 3D rabbit polyclonal antibodies have been described, anti-poliovirus capsid conformation epitope humanized antibody A12 has also been described. Anti-NDV NP protein rabbit polyclonal antibodies were described previously, anti-NDV HN mouse monoclonal antibodies were obtained separately. Anti-actin mouse monoclonal antibody conjugated with horseradish peroxidase (HRP) was from Sigma Aldrich. Secondary antibody conjugates with Alexa fluorescent dyes were from Molecular Probes. HRP-conjugated rabbit anti-guinea pig IgM, goat anti-guinea pig IgG and sheep anti-guinea pig IgA antibodies from were from Bioss, Invitrogen and ICL, respectively.

Immunofluorescence Assay.

For immunofluorescence cells grown on a coverslip were fixed with 4% formaldehyde in PBS for 20 min, washed with PBS and permeabilized with 0.2% Triton-X100 for 5 min. Primary and secondary antibodies were diluted in PBS with 3% ECL blocking reagent (Amersham), the same solution was used for blocking of non-specific antibody binding sites. Images were taken with Zeiss Axiovert 200M epifluorescent microscope equipped with a digital camera. Digital images were processed using Adobe Photoshop software, the same parameters were applied to the whole image area.

Polio Replicon Replication Assay.

The replication assay was performed essentially according to known techniques, with the exception that instead of replicon RNA transfection HeLa cells were infected with replicon RNA transencapsidated in poliovirus capsid proteins expressed during replication of NDV-polio recombinant virus. Briefly, HeLa cells grown in 96-well plate were incubated with lysates of cells from transencapsidation experiment for 1 h, washed and incubated in growth medium supplemented with 30 μM of cell-permeable Renilla luciferase substrate EnduRen (Promega). The measurements of Renilla luciferase activity indicative of polio RNA replication were performed automatically every hour using TECAN M1000 plate reader.

Next Generation Sequencing.

NDV-polio propagated in embryonated chicken eggs was purified from allantoic fluid by centrifugation through 30% sucrose cushion in PBS at 75000 g for 2.5 h. The viral RNA was isolated with QiaAmp viral RNA isolation kit (Qiagen) using a protocol without carrier RNA. To prepare 250 nucleotide-long DNA library for IIlumina sequencing, 0.5 μg of total RNA was fragmented using a focused-ultrasonicator (Covaris) to generate fragments 300 to 500 nt of size. The DNA libraries from the RNA fragments were prepared using the NEBNext mRNA Library Prep Master Mix Set for IIlumina (New England BioLabs) according to the manufacturer's protocol, briefly; the fragmented RNA was reverse transcribed and the DNA second strand was synthesized. The resulted DNA fragments were ligated to IIlumina paired end (PE) adaptors, then amplified using 12 cycles of PCR with multiplex indexed primers and purified by magnetic beads (Agencourt AMPure PCR purification system, BeckmanCoulter). After analyzing the DNA libraries for size and quality (BioAnalyzer, Agilent Technologies, Inc.), deep sequencing was performed using MiSeq (Illumina) producing 250 nucleotide paired-end reads. The raw sequencing reads were analyzed by the HIVE software package; the data are deposited at hive.biochemistry.gwu.edu/review/NDV_LaSota_strain_PV.

Immunization of Guinea Pigs.

All animals were handled in accordance with established guidelines and the procedures were performed according to the University of Maryland IACUC-approved protocol. Five week old female Hartley guinea pigs were divided into 2 groups of 4 animals each which were immunized with either recombinant NDV-polio virus or vector NDV without poliovirus inserts with 10E5 PFU of each virus. Allantoic fluid collected from one egg provides from ^{˜}500 to ^{˜}1000 such immunization doses. For immunization allantoic fluid diluted in PBS was administered intranasally (100 μl/nostril). Boost immunization was performed similarly at 3 weeks after first immunization. Blood from thoracic vein was collected at weeks 0 (prebleed), 3 (before the boost immunization), 5, 7, 9 and 11. Vaginal washes were collected at the same time as the blood samples. Animal feeding needles (Fisher Scientific) were used to flush 100 μl of PBS containing protease inhibitor cocktail (Sigma) 4-6 times into vaginal cavity. Vaginal washes were clarified by centrifugation at 10,000 rpm for 15 min in a minifuge to remove cellular debris, and supernatants were collected and stored at −70 C. Prior to antibody assay and microneutralization test, the protein concentration of vaginal washes was determined by Bradford protocol. The samples with protein concentration lower than 0.4 μg/μl were excluded from the analysis.

Poliovirus Microneutralization Assay.

Titer of anti-poliovirus neutralizing antibodies in serum and vaginal washes of immunized guinea pigs was determined using microneutralization assay in 96-well plate format. Briefly, heat-inactivated serum, or vaginal washes adjusted to the same protein concentration with PBS, and poliovirus type I Mahoney were diluted in DMEM supplemented with 2% FBS. 50 μl of serial serum dilutions were incubated with 50 μl of medium containing 100 TCID₅₀ of poliovirus for 3 h, after that 50 μl of HeLa cells suspension (30000 cells/well) were added, and the plate was incubated for 4 days in cell culture incubator. Staining with crystal violet was performed at the end of incubation period to determine virus-induced CPE. All experiments included virus titration control and neutralization control with in-house standard rabbit anti-poliovirus serum.

Anti-NDV IgG Assay.

For assessment of seum anti-NDV IgG antibodies a commercial NDV Antibody Test Kit from AffiniTech was used. Briefly, serial dilutions of sera in PBS were incubated in 96-well plates with pre-adsorbed NDV antigens. The plates were processed for colorimetric assay using HRP-linked goat anti-guinea pig IgG antibodies and reagents provided in the kit with ABTS (2,2′-azinobis [3-ethylbenzothiazoline-6-sulfonic acid]-diammonium salt) peroxidase substrate solution (Synbiotics Carporation), according to the manufacturer's protocol. The highest serum dilution producing signal more than two times that of the pre-bleed control serum is plotted.

Anti-Poliovirus IgM, IgA and IgG Assay.

For assessment of serum anti-poliovirus IgM and IgA and anti-poliovirus IgG in vaginal washes antibodies 10 μl of type one inactivated poliovirus vaccine (IPV) (containing formalin-inactivated whole virions) diluted 1:100 in PBS was spotted onto nitrocellulose membrane using 96-well manifold (Schleicher & Shull). The non-specific binding sites were blocked by one hour incubation in 2% ECL blocking reagent (Amersham) in tris-buffered saline supplemented with 0.002% Tween-20 (TBS-T). The same solution was used for serial serum dilutions, for dilutions of vaginal washes previously adjusted to the same protein concentration with PBS, and dilution of secondary anti-guinea pig antibodies conjugated with horseradish peroxidase. The membrane was developed using ECL Select detection reagent (GE Healthcare) and the signal was recorded using Azure C300 chemiluminescent reader. The signal was quantified using Image Studio Lite software (LiCore). The highest serum dilution producing signal more than two times that of the pre-bleed control serum is plotted.

Ethics Statement.

Animal protocols were designed according to the Guide for the Care and Use of Laboratory Animals. The guinea pig immunization protocol R-16-57 was approved by the University of Maryland, College Park Institutional Animal Care and Use Committee (IACUC), IBC #14-12. Hartley guinea pigs and 9 day-old embryonated chicken eggs were purchased from Charles River laboratories.

### Discussion

It will be apparent from the foregoing data that this disclosure provides an alternative approach to designing a safe and effective anti-poliovirus vaccine. We introduced sequences coding for poliovirus capsid protein precursor P1 and protease 3CD required for its processing in the genome of NDV, a negative strand RNA virus with mucosal tropism. We reasoned that such an approach would address the major hurdles in the development of a next generation anti-poliovirus vaccine. First, the vectored vaccine is safe in terms of re-introduction of poliovirus in the environment, since the construct contains only fragments of poliovirus genome, and is in principle incapable of recombination with circulating enteroviruses. Second, poliovirus proteins are expressed and assemble into VLPs in vivo, upon replication of the recombinant virus, thus eliminating the problem of isolation and stabilization of labile VLPs. Indeed, the presently provided data show that poliovirus capsid proteins produced in NDV-polio infected cells assemble into VLPs with the antigenic structure of poliovirus virions. They were recognized by A12 antibody that binds a conformational epitope present only in functional virions, they were capable to transencapsidate a replicon RNA into functional virions, and they were capable of inducing neutralizing antibody response. Finally, poliovirus VLPs are presented to the immune system in the context of active NDV replication in mucosal surface, serving as natural adjuvant and stimulating the development of both systemic and mucosal immune response. Furthermore, NDV-based vaccines are delivered via intranasal spray, thus retaining one of the major advantages of live poliovirus vaccine which is administered orally by personnel without medical training.

Further, data presented herein demonstrate robust development of neutralizing anti-poliovirus antibodies upon two intranasal immunizations of guinea pigs. We chose guinea pigs as a model for evaluation of this vaccine over mice expressing poliovirus receptor for several reasons. While mice expressing poliovirus receptor are available, they are susceptible to oral poliovirus infection necessary to evaluate the development of gut mucosal immunity only in the background of interferon A/B receptor knockout, thus their immune response to vaccination cannot be informative. Mice expressing poliovirus receptor in the wt background can only be infected with poliovirus via intramuscular, intraperitoneal or direct inoculation into the CNS and essentially allow measurement of neutralizing antibody in the serum, a parameter that can be quantitatively assessed in the microneutralization assay. Guinea pigs, on the other hand, due to their bigger size allow better control of intranasal vaccine delivery and collection of larger blood samples necessary to thorough evaluate the immune response. Moreover, the immune response of guinea pigs to immunization with NDV-based vaccines was previously shown to better recapitulate that observed in primate models and to include a strong mucosal component. Due to the nature of the guinea pig model, we did not determine if the representative vaccine of this disclosure was capable of inducing gut immunity sufficient to interrupt poliovirus transmission. However, we observed accumulation of anti-poliovirus IgM and IgA antibodies in the blood serum, as well as anti-poliovirus neutralizing and specific IgG antibodies in vaginal washes, strongly indicative of the development of protective mucosal immune response. The mucosal membranes in the body are involved in constant immunological communication and in many cases may be considered as a unified immunological site. Indeed, recent research indicates that intranasal administration of different antigens can elicit gut mucosal immunity, including the development of protective response against human Norwalk virus, an enteric pathogen with a life cycle similar to that of poliovirus.

Two considerations are related to development of a live vectored vaccine. First, the pre-existing immunity to the vector should not interfere with vaccine performance. Second, the vector itself should be safe not only to vaccine recipients, but there should be no safety concern in case of its inevitable release into the environment. Humans generally do not have pre-existing immunity to NDV, although sporadic cases of NDV infections have been described in poultry industry workers or people handling infected birds. The infections were quickly self-resolving and either asymptomatic or manifested as mild conjunctivitis or laryngitis, and no cases of human to human transmission have been observed. Moreover, NDV has been explored as an oncolytic virus, and demonstrated excellent safety profile in clinical trials, even when high doses of the virus were administered intravenously to individuals with a compromised immune system. NDV and related viruses infect a broad spectrum of bird species, and pathogenic strains of NDV can cause devastating epizootics in poultry. In this disclosure we utilized a non-pathogenic LaSota strain of NDV, which is broadly used as a live vaccine in the poultry industry and has never been documented to develop pathogenic phenotype. Thus, production and administration of a vectored vaccine based on LaSota backbone should not raise any safety concerns. The virus replicates to high titers in embryonated chicken eggs, an established and one of the most effective systems for vaccine production.

An important property of the viruses of the order Mononegavirales that includes NDV is the practically non-existent recombination, leading to a much higher genome stability than that of other groups of RNA viruses. In the present analysis of the stability of the poliovirus inserts upon propagation of plaque-purified NDV-polio viruses we did not observe any significant changes in poliovirus protein expression, neither NGS analysis of the viral RNA revealed accumulation of mutations during at least 10 passages in emblyonated eggs. It should be noted that each passage in eggs entails multiple cycles of viral replication. Nevertheless, production of modern vaccines employs a seed virus system that only allows manufacturing process involving one or two passages from the seed virus stock. Therefore, stability of a vectored NDV-based anti-poliovirus vaccine should be sufficient for industrial propagation.

To develop vaccines covering all three poliovirus serotypes, the present disclosure includes using other antigenically distinct avian paramixovirus (APMV) types as backbones for expression of poliovirus VLPs of different serotypes. This will allow administration of vaccines against all three serotypes of poliovirus or other viruses in different combinations, or in any temporal succession, so that one vectored vaccine does not interfere with the performance of the other. The advantage of APMV as a vector is that there are at least 15 APMV types with properties similar to that of NDV (APMV type 1), that are not pathogenic to poultry, which can be explored as backbones for vectored vaccines. Even though the presently described construct expressed poliovirus proteins to the level similar to that observed in poliovirus-infected cells, it is realistic to increase it even further. For example, the disclosure includes optimizing translation initiation capacity of the RNA coding for, for example, poliovirus capsid protein precursor, the other is to place the polio capsid insert closer to the 3′end of the NDV genome, between N and P genes of NDV to increase the level of transcription. Collectively, these data indicate that NDV is a highly promising vector for the development of a safe, effective and affordable anti-poliovirus vaccine.

Representative sequences used in embodiments of this disclosure:
Poliovirus type I Mahoney P1 insert (italics) in the LaSota vector sequence (inserted into PmeI site), given as DNA sequence:

(SEQ ID NO: 1)
aaacttagaaaaaatacgggtagaatcggccggccgccaccatgggtg
ctcaggtttcatcacagaaagtgggcgcacatgaaaactcaaatagag
cgtatggtggttctaccattaattacaccaccattaattattatagag
attcagctagtaacgcggcttcgaaacaggacttctctcaagaccctt
ccaagttcaccgagcccatcaaggatgtcctgataaaaacagccccaa
tgctaaactcgccaaacatagaggcttgcgggtatagcgatagagtac
tgcaattaacactgggaaactccactataaccacacaggaggcggcta
attcagtagtcgcttatgggcgttggcctgaatatctgagggacagcg
aagccaatccagtggaccagccgacagaaccagacgtcgctgcatgca
ggttttatacgctagacaccgtgtcttggacgaaagagtcgcgagggt
ggtggtggaagttgcctgatgcactgagggacatgggactctttgggc
aaaatatgtactaccactacctaggtaggtccgggtacaccgtgcatg
tacagtgtaacgcctccaaattccaccagggggcactaggggtattcg
ccgtaccagagatgtgtctggccggggatagcaacaccactaccatgc
acaccagctatcaaaatgccaatcctggcgagaaaggaggcactttca
cgggtacgttcactcctgacaacaaccagacatcacctgcccgcaggt
tctgcccggtggattacctccttggaaatggcacgttgttggggaatg
cctttgtgttcccgcaccagataataaacctacggaccaacaactgtg
ctacactggtactcccttacgtgaactccctctcgatagatagtatgg
taaagcacaataattggggaattgcaatattaccattggccccattaa
attttgctagtgagtcctccccagagattccaatcaccttgaccatag
cccctatgtgctgtgagttcaatggattaagaaacatcaccctgccac
gcttacagggcctgccggtcatgaacacccctggtagcaatcaatatc
ttactgcagacaacttccagtcaccgtgtgcgctgcctgaatttgatg
tgaccccacctattgacatacccggtgaagtaaagaacatgatggaat
tggcagaaatcgacaccatgattccctttgacttaagtgccacaaaaa
agaacaccatggaaatgtatagggttcggttaagtgacaaaccacata
cagacgatcccatactctgcctgtcactctctccagcttcagatccta
ggttgtcacatactatgcttggagaaatcctaaattactacacacact
gggcaggatccctgaagttcacgtttctgttctgtggattcatgatgg
caactggcaaactgttggtgtcatacgcgcctcctggagccgacccac
caaagaagcgtaaggaggcgatgttgggaacacatgtgatctgggaca
taggactgcagtcctcatgtactatggtagtgccatggattagcaaca
ccacgtatcggcaaaccatagatgatagtttcaccgaaggcggataca
tcagcgtcttctaccaaactagaatagtcgtccctctttcgacaccca
gagagatggacatccttggttttgtgtcagcgtgtaatgacttcagcg
tgcgcttgttgcgagataccacacatatagagcaaaaagcgctagcac
aggggttaggtcagatgcttgaaagcatgattgacaacacagtccgtg
aaacggtgggggcggcaacatctagagacgctctcccaaacactgaag
ccagtggaccaacacactccaaggaaattccggcactcaccgcagtgg
aaactggggccacaaatccactagtcccttctgatacagtgcaaacca
gacatgttgtacaacataggtcaaggtcagagtctagcatagagtctt
tcttcgcgcggggtgcatgcgtgaccattatgaccgtggataacccag
cttccaccacgaataaggataagctatttgcagtgtggaagatcactt
ataaagatactgtccagttacggaggaaattggagttcttcacctatt
ctagatttgatatggaacttacctttgtggttactgcaaatttcactg
agactaacaatgggcatgccttaaatcaagtgtaccaaattatgtacg
taccaccaggcgctccagtgcccgagaaatgggacgactacacatggc
aaacctcatcaaatccatcaatcttttacacctacggaacagctccag
cccggatctcggtaccgtatgttggtatttcgaacgcctattcacact
tttacgacggtttttccaaagtaccactgaaggaccagtcggcagcac
taggtgactccctttatggtgcagcatctctaaatgacttcggtattt
tggctgttagagtagtcaatgatcacaacccgaccaaggtcacctcca
aaatcagagtgtatctaaaacccaaacacatcagagtctggtgcccgc
gtccaccgagggcagtggcgtactacggccctggagtggattacaagg
atggtacgcttacacccctctccaccaaggatctgaccacatattagt
taacgttt

Poliovirus type I Mahoney 3CD insert (italics) in the LaSota vector sequence (inserted into SnaBI site), given as DNA sequence:

(SEQ ID NO: 2)
gtaacgggtagaacgttggcgcgccgccaccatgggaccagggttcga
ttacgcagtggctatggctaaaagaaacattgttacagcaactactag
caagggagagttcactatgttaggagtccacgacaacgtggctatttt
accaacccacgcttcacctggtgaaagcattgtgatcgatggcaaaga
agtggagatcttggatgccaaagcgctcgaagatcaagcaggaaccaa
tcttgaaatcactataatcactctaaagagaaatgaaaagttcagaga
cattagaccacatatacctactcaaatcactgagacaaatgatggagt
cttgatcgtgaacactagcaagtaccccaatatgtatgttcctgtcgg
tgctgtgactgaacagggatatctaaatctcggtgggcgccaaactgc
tcgtactctaatgtacaactttccaaccagagcaggacagtgtggtgg
agtcatcacatgtactgggaaagtcatcgggatgcatgttggtgggaa
cggttcacacgggtttgcagcggccctgaagcgatcatacttcactca
gagtcaaggtgaaatccagtggatgagaccttcgaaggaagtgggata
tccaatcataaatgccccgtccaaaaccaagcttgaacccagtgcttt
ccactatgtgtttgaaggggtgaaggaaccagcagtcctcactaaaaa
cgatcccaggcttaagacagactttgaggaggcaattttctccaagta
cgtgggtaacaaaattactgaagtggatgagtacatgaaagaggcagt
agaccactatgctggccagctcatgtcactagacatcaacacagaaca
aatgtgcttggaggatgccatgtatggcactgatggtctagaagcact
tgatttgtccaccagtgctggctacccttatgtagcaatgggaaagaa
gaagagagacatcttgaacaaacaaaccagagacactaaggaaatgca
aaaactgctcgacacatatggaatcaacctcccactggtgacttatgt
aaaggatgaacttagatccaaaacaaaggttgagcaggggaaatccag
attaattgaagcttctagtttgaatgactcagtggcaatgagaatggc
ttttgggaacctatatgctgcttttcacaaaaacccaggagtgataac
aggttcagcagtggggtgcgatccagatttgttttggagcaaaattcc
ggtattgatggaagagaagctgtttgcttttgactacacagggtatga
tgcatctctcagccctgcttggttcgaggcactaaagatggtgcttga
gaaaatcggattcggagacagagttgactacatcgactacctaaacca
ctcacaccacctgtacaagaataaaacatactgtgtcaagggcggtat
gccatctggctgctcaggcacttcaatttttaactcaatgattaacaa
cttgattatcaggacactcttactgaaaacctacaagggcatagattt
agaccacctaaaaatgattgcctatggtgatgatgtaattgcttccta
cccccatgaagttgacgctagtctcctagcccaatcaggaaaagacta
tggactaactatgactccagctgacaaatcagctacatttgaaacagt
cacatgggagaatgtaacattcttgaagagattcttcagggcagacga
gaaatacccatttcttattcatccagtaatgccaatgaaggaaattca
tgaatcaattagatggactaaagatcctaggaacactcaggatcacgt
tcgctctctgtgccttttagcttggcacaatggcgaagaagaatataa
caaattcctagctaaaatcaggagtgtgccaattggaagagctttatt
gctcccagagtactcaacattgtaccgccgttggcttgactcatttta
ggcgatcgcttagaaaaaatac

Poliovirus inserts correspond to: GenBank: V01148.1, from which the sequence is incorporated herein by reference as it exists in the database as of the priority date of this application or patent: P1 insert: nt 744-3380; 3CD insert: nt 5433-7364.

The foregoing examples are intended to illustrate, but not limit the scope of this disclosure.

We claim:

1. A modified Newcastle Disease virus (NDV) that produces virus-like particles (VLPs) in a host recipient, the modified NDV comprising:
a single stranded negative sense RNA polynucleotide comprising nucleotide sequences configured in a 3′-5′ direction encoding NDV nucleocapsid protein (NP), phosphoprotein (P), matrix protein (M), fusion protein (F), hemagglutinin-neuraminidase (HN) and RNA-dependent RNA polymerase (L) protein, and:
i) a first nucleotide sequence encoding a picornavirus capsid polyprotein precursor, the first nucleotide sequence positioned between the between P and M nucleotide sequences,
and
ii) a second nucleotide sequence encoding a picornavirus protease that is capable of processing the capsid polyprotein precursor into a capsid protein such that the capsid protein is incorporated into the VLPs, wherein the second nucleotide sequence is positioned between the HN and L nucleotide sequences.

2. The modified NDV of claim 1, wherein the modified NDV comprises an infectious non-pathogenic viral particle.

3. The modified NDV of claim 1, wherein the first and second nucleotide sequences are each flanked by NDV transcription start and stop signals.

4. The modified NDV of claim 1, wherein the modified NDV has mucosal tropism.

5. The modified NDV of claim 1, wherein the modified NDV comprises a LaSota strain of NDV.

6. The modified NDV of claim 1, wherein the capsid polyprotein precursor and the protease are from a poliovirus.

7. The modified NDV of claim 6, wherein the capsid polyprotein precursor comprises poliovirus type I Mahoney capsid protein precursor P1, and wherein the protease comprises poliovirus protease 3CD.

8. A composition comprising a pharmaceutically acceptable additive and purified infectious, non-pathogenic modified Newcastle Disease virus (NDV) viral particles comprising the single stranded negative sense RNA polynucleotide of claim 1.

9. The composition of claim 8, wherein the composition is free of adjuvant.

10. A method of stimulating an immune response against a picornavirus capsid protein comprising administering the composition of claim 8 to a mammal or an avian animal in need thereof.

11. The method of claim 10, wherein the composition is free of adjuvant.

12. The method of claim 10, wherein the administration produces neutralizing antibodies that bind with specificity to the capsid protein.

13. The method of claim 10, wherein the administration comprises a mucosal administration.

14. The method of claim 13, wherein the mucosal administration comprises an intranasal administration.

15. The method of claim 10, wherein the mammal is a human.

16. The method of claim 10, wherein the mammal is a porcine mammal, a bovine mammal, an equine mammal, a canine mammal, or a feline mammal.

17. The method of claim 10, wherein the composition is administered to the avian animal.

18. A kit for use in making the NDV of claim 1.


SEQUENCE LISTING (2 sequences)

SEQ ID NO: 1 - DNA, 2696 bp, artificial sequence
  Recombinant LaSota vector cDNA sequence
aaacttagaaaaaatacgggtagaatcggccggccgccaccatgggtgctcaggtttcatcacagaaagtgggcgcacatgaaaactcaaatagagcgtatggtggttctaccattaattacaccaccattaattattatagagattcagctagtaacgcggcttcgaaacaggacttctctcaagacccttccaagttcaccgagcccatcaaggatgtcctgataaaaacagccccaatgctaaactcgccaaacatagaggcttgcgggtatagcgatagagtactgcaattaacactgggaaactccactataaccacacaggaggcggctaattcagtagtcgcttatgggcgttggcctgaatatctgagggacagcgaagccaatccagtggaccagccgacagaaccagacgtcgctgcatgcaggttttatacgctagacaccgtgtcttggacgaaagagtcgcgagggtggtggtggaagttgcctgatgcactgagggacatgggactctttgggcaaaatatgtactaccactacctaggtaggtccgggtacaccgtgcatgtacagtgtaacgcctccaaattccaccagggggcactaggggtattcgccgtaccagagatgtgtctggccggggatagcaacaccactaccatgcacaccagctatcaaaatgccaatcctggcgagaaaggaggcactttcacgggtacgttcactcctgacaacaaccagacatcacctgcccgcaggttctgcccggtggattacctccttggaaatggcacgttgttggggaatgcctttgtgttcccgcaccagataataaacctacggaccaacaactgtgctacactggtactcccttacgtgaactccctctcgatagatagtatggtaaagcacaataattggggaattgcaatattaccattggccccattaaattttgctagtgagtcctccccagagattccaatcaccttgaccatagcccctatgtgctgtgagttcaatggattaagaaacatcaccctgccacgcttacagggcctgccggtcatgaacacccctggtagcaatcaatatcttactgcagacaacttccagtcaccgtgtgcgctgcctgaatttgatgtgaccccacctattgacatacccggtgaagtaaagaacatgatggaattggcagaaatcgacaccatgattccctttgacttaagtgccacaaaaaagaacaccatggaaatgtatagggttcggttaagtgacaaaccacatacagacgatcccatactctgcctgtcactctctccagcttcagatcctaggttgtcacatactatgcttggagaaatcctaaattactacacacactgggcaggatccctgaagttcacgtttctgttctgtggattcatgatggcaactggcaaactgttggtgtcatacgcgcctcctggagccgacccaccaaagaagcgtaaggaggcgatgttgggaacacatgtgatctgggacataggactgcagtcctcatgtactatggtagtgccatggattagcaacaccacgtatcggcaaaccatagatgatagtttcaccgaaggcggatacatcagcgtcttctaccaaactagaatagtcgtccctctttcgacacccagagagatggacatccttggttttgtgtcagcgtgtaatgacttcagcgtgcgcttgttgcgagataccacacatatagagcaaaaagcgctagcacaggggttaggtcagatgcttgaaagcatgattgacaacacagtccgtgaaacggtgggggcggcaacatctagagacgctctcccaaacactgaagccagtggaccaacacactccaaggaaattccggcactcaccgcagtggaaactggggccacaaatccactagtcccttctgatacagtgcaaaccagacatgttgtacaacataggtcaaggtcagagtctagcatagagtctttcttcgcgcggggtgcatgcgtgaccattatgaccgtggataacccagcttccaccacgaataaggataagctatttgcagtgtggaagatcacttataaagatactgtccagttacggaggaaattggagttcttcacctattctagatttgatatggaacttacctttgtggttactgcaaatttcactgagactaacaatgggcatgccttaaatcaagtgtaccaaattatgtacgtaccaccaggcgctccagtgcccgagaaatgggacgactacacatggcaaacctcatcaaatccatcaatcttttacacctacggaacagctccagcccggatctcggtaccgtatgttggtatttcgaacgcctattcacacttttacgacggtttttccaaagtaccactgaaggaccagtcggcagcactaggtgactccctttatggtgcagcatctctaaatgacttcggtattttggctgttagagtagtcaatgatcacaacccgaccaaggtcacctccaaaatcagagtgtatctaaaacccaaacacatcagagtctggtgcccgcgtccaccgagggcagtggcgtactacggccctggagtggattacaaggatggtacgcttacacccctctccaccaaggatctgaccacatattagttaacgttt

SEQ ID NO: 2 - DNA, 1990 bp, artificial sequence
  Poliovirus type I Mahoney 3CD insert in recombinant LaSota vector
gtaacgggtagaacgttggcgcgccgccaccatgggaccagggttcgattacgcagtggctatggctaaaagaaacattgttacagcaactactagcaagggagagttcactatgttaggagtccacgacaacgtggctattttaccaacccacgcttcacctggtgaaagcattgtgatcgatggcaaagaagtggagatcttggatgccaaagcgctcgaagatcaagcaggaaccaatcttgaaatcactataatcactctaaagagaaatgaaaagttcagagacattagaccacatatacctactcaaatcactgagacaaatgatggagtcttgatcgtgaacactagcaagtaccccaatatgtatgttcctgtcggtgctgtgactgaacagggatatctaaatctcggtgggcgccaaactgctcgtactctaatgtacaactttccaaccagagcaggacagtgtggtggagtcatcacatgtactgggaaagtcatcgggatgcatgttggtgggaacggttcacacgggtttgcagcggccctgaagcgatcatacttcactcagagtcaaggtgaaatccagtggatgagaccttcgaaggaagtgggatatccaatcataaatgccccgtccaaaaccaagcttgaacccagtgctttccactatgtgtttgaaggggtgaaggaaccagcagtcctcactaaaaacgatcccaggcttaagacagactttgaggaggcaattttctccaagtacgtgggtaacaaaattactgaagtggatgagtacatgaaagaggcagtagaccactatgctggccagctcatgtcactagacatcaacacagaacaaatgtgcttggaggatgccatgtatggcactgatggtctagaagcacttgatttgtccaccagtgctggctacccttatgtagcaatgggaaagaagaagagagacatcttgaacaaacaaaccagagacactaaggaaatgcaaaaactgctcgacacatatggaatcaacctcccactggtgacttatgtaaaggatgaacttagatccaaaacaaaggttgagcaggggaaatccagattaattgaagcttctagtttgaatgactcagtggcaatgagaatggcttttgggaacctatatgctgcttttcacaaaaacccaggagtgataacaggttcagcagtggggtgcgatccagatttgttttggagcaaaattccggtattgatggaagagaagctgtttgcttttgactacacagggtatgatgcatctctcagccctgcttggttcgaggcactaaagatggtgcttgagaaaatcggattcggagacagagttgactacatcgactacctaaaccactcacaccacctgtacaagaataaaacatactgtgtcaagggcggtatgccatctggctgctcaggcacttcaatttttaactcaatgattaacaacttgattatcaggacactcttactgaaaacctacaagggcatagatttagaccacctaaaaatgattgcctatggtgatgatgtaattgcttcctacccccatgaagttgacgctagtctcctagcccaatcaggaaaagactatggactaactatgactccagctgacaaatcagctacatttgaaacagtcacatgggagaatgtaacattcttgaagagattcttcagggcagacgagaaatacccatttcttattcatccagtaatgccaatgaaggaaattcatgaatcaattagatggactaaagatcctaggaacactcaggatcacgttcgctctctgtgccttttagcttggcacaatggcgaagaagaatataacaaattcctagctaaaatcaggagtgtgccaattggaagagctttattgctcccagagtactcaacattgtaccgccgttggcttgactcattttaggcgatcgcttagaaaaaatac